(19) United States
(12) Patent Application Publication
(10) Pub. No.: US 2008/0312447 A1
(43) Pub. Date: Dec. 18, 2008

(54) Process for Isolation of Crystalline Tacrolimus

(75) Inventors: Ladislav Cvak, Opava (CZ); Alexandr Jegorov, Dobra Voda (CZ); Martin Buchta, Ludgerovice (CZ); Pavel Blatny, Opava (CZ); Josef Satke, Opava (CZ)

Correspondence Address: LERNER, DAVID, LITTENBERG,;KRUMHOLZ & MENTLIK, 600 SOUTH AVENUE WEST, WESTFIELD, NJ, 07090, US

(73) Assignee: IVAX Pharmaceuticals s.r.o., Opava 9 (CZ)

(21) Appl. No.: 11/662,235

(22) PCT Filed: Sep. 9, 2005
(86) PCT No.: PCT/US05/32258

§ 371 Date: Apr. 1, 2008

Related U.S. Application Data
(60) Provisional application No. 60/608,752, filed on Sep. 10, 2004.
(60) Provisional application No. 60/608,757, filed on Sep. 10, 2004.

(51) Int. Cl. C07D 407/06

(52) U.S. Cl. 546/207

(57) ABSTRACT

The invention is a simple process for separation of tacrolimus and its analogues, ascomycin and tsucubamycin B and preparation of enough pure crystalline tacrolimus. The process takes advantage of surprising properties of tacrolimus and involves extraction, purification and crystallization to produce purified crystalline tacrolimus.

### FIELD OF THE INVENTION

The present invention relates to a process for isolation of crystalline tacrolimus from the fermentation broth.

### BACKGROUND OF THE INVENTION

Tacrolimus, also known as FK 506, is a naturally occurring macrolide antibiotic with selective inhibitory effect on T-lymphocytes. It is used as an immunosuppressive drug. Tacrolimus was first described in patents, e.g., U.S. Pat. No. 4,894,366 and EP 184,162. Later it was also described in the scientific papers: H. Tanaka et al. J. Am. Chem. Soc. 1987, 109, 5031-5033 and T. Kino et al. J. Antibiot. 1987,40,1249-1255.

Preferred process for tacrolimus preparation is fermentation, although its total synthesis was also described, e.g., in EP 378,318. Isolation of tacrolimus from the fermentation broth is relatively difficult. Unfortunately, most tacrolimus producing strains, produce also ascomycin and some other macrolide compounds, e.g., tsucubamycin B, therefore, the separation of tacrolimus and other macrolides must be involved in the process for the isolation of pure tacrolimus. Another difficulty of tacrolimus isolation is its low concentration in the biomass and the fact that the compound is usually present in both the solid phase (mycelium) and the liquid phase (filtered fermentation broth). Hence, the process for economical isolation of tacrolimus requires the separation of the mycelium and separated processing of both the mycelium and the filtered fermentation broth, as described, e.g., in T. Kino et al. J. Antibiot. 1987, 40, 1249-1255. Another possibility is described in patent application WO 03/68 980, claiming direct extraction of the whole fermentation broth with a hydrophobic organic solvents.

Ascomycin and tsucubamycin B are natural analogues of tacrolimus: while tacrolimus contains allyl group in the position 21 of the macrolide skeleton, ascomycin has there an ethyl group and tsucubamycin B a propyl group as described, e.g., by H. Hatanaka et al. (J. Antibiot. 1988,41,1592-1599) and M. Morisaki at al. (J. Antibiot. 1992,45,126-132). Other natural derivatives and analogues of tacrolimus were described in patents, e.g., EP 358,508 and GB 2,269,172.

Separation of tacrolimus and other macrolide antibiotics (macrolides), namely ascomycin and tsucubamycin B is very difficult due to the similarity of the compounds. Satisfactory separation is not possible by any crystallization reported so far. The only possibility is then a column chromatography. Numerous HPLC methods have been described in the literature, all of them use reverse phase system, e.g., Y. Nakimi et al. Chromatographia 1995, 40,253-258, T. Nishikawa et al. Pharm. Res. 1993,10,1785-1789, T. Akashi et al. J. Pharm. Biomed. Anal. 1996, 14, 339-346. Reverse phase systems are not convenient for preparative chromatography because of their water containing mobile phase facilitates isomerization of tacrolimus to its tautomers: Y. Nakimi et al. Chromatographia 1995,40, 253-258. Moreover, the isolation of tacrolimus from the eluate is also inconvenient.

Another possibility for separation of tacrolimus and ascomycin and tsucubamycin B was described in patent application WO 01/18 007, where a reverse phase chromatography with mobile phase containing silver ions was used. Nevertheless the use of water containing mobile phase is still the drawbacks of the process.

### SUMMARY OF THE INVENTION

The process according to the invention offers a simple process for isolating very pure tacrolimus from the fermentation broth in a high yield. The extraction of the macrolides from the mycelium is accomplished by the addition of a suitable water miscible organic solvent to the whole fermentation broth. The mixture of macrolides is thus transferred to the liquid phase. The extracted mycelium is then separated and the liquid phase (the aqueous extract) is further processed by extraction with a suitable water non miscible solvent, obtaining thus the organic extract. The organic extract is then partially evaporated, obtaining a tacrolimus concentrate. The tacrolimus concentrate is further purified by a chromatography on a silica gel modified by a salt of silver, using suitable organic solvent as a mobile phase. The fractions containing enough pure tacrolimus are then concentrated and the residue is crystallized from a suitable solvent, obtaining pure crystalline tacrolimus.

In another embodiment of the process, the aqueous extract can be prepared by extraction of the separated mycelium containing tacrolimus with a mixture of water and water miscible organic solvent. The further processing of the aqueous extract is the same as described above.

In another embodiment of the process, the aqueous extract is not separated from the mycelium before subjecting to the treatment with a water non miscible solvent. The water non miscible solvent can be added directly to the suspension of mycelium in aqueous extract and the organic extract can be then separated from the three phase system.

In another embodiment of the process, another chromatographic step can be immerged before the chromatographic purification on a silica gel modified with a silver salt. In this step the tacrolimus concentrate is purified on a silica gel using a suitable organic solvent as the mobile phase and obtained fraction containing tacrolimus and other macrolide compounds are further purified on a silica gel modified with silver salt.

### DESCRIPTION OF THE DRAWINGS

FIG. 1. Preparative chromatography of the tacrolimus concentrate on a silica gel using mixture of toluene and acetone 85:15 (v/v) as the mobile phase (reconstruction based on HPLC analysis of fractions).

FIG. 2. Preparative chromatography of the tacrolimus concentrate on a silica gel modified with silver nitrate (prepared according to example 3), using mixture of toluene and acetone 85:15 (v/v) as the mobile phase (reconstruction based on HPLC analysis of fractions).

FIG. 3. HPLC analysis of crystalline tacrolimus obtained in Example 1.

FIG. 4. HPLC analysis of the residue after first chromatography obtained in Example 2.

FIG. 5. HPLC analysis of crystalline tacrolimus obtained in Example 2.

### DETAILED DESCRIPTION OF THE INVENTION

Although tacrolimus is insoluble in water, surprisingly high part of tacrolimus was found in the liquid phase of the fermentation broth, especially when the total production of the fermentation process was low. Therefore, the processing of the whole fermentation broth, that is, the suspension obtained by the cultivation of a microorganism producing tacrolimus, is highly advisable. The process according to the invention is capable to process the whole fermentation broth, using cheap and environmentally acceptable solvents. Adding a suitable water miscible organic solvent to the whole fermentation broth leads to the extraction of a mixture of macrolides into the liquid phase. Such water miscible solvents can be lower aliphatic alcohols or ketones. Preferable solvents are acetone, 2-propanol, or 1-propanol. On the other side, methanol is not convenient due to its high reactivity, which contributes to the decomposition of tacrolimus. The reactivity of ethanol is substantially lower than that of methanol, nevertheless, it is not negligible and therefore, ethanol can be used for extraction of macrolide compounds, but it is less convenient than the above mentioned solvents acetone, 1-propanol, and/or 2-propanol.

The aqueous extract obtained by adding of a water miscible organic solvent to the whole fermentation broth can be separated from the extracted mycelium by filtration or by sedimentation, preferably by centrifugal separation, and the obtained clear aqueous extract is further processed without any evaporation. Second possibility is to process the aqueous extract without separation of the solid phase.

Another possibility, how to prepare the aqueous extract of tacrolimus, is the extraction of the separated mycelium with a mixture of water and a water miscible solvent. This attitude can be convenient mainly when a fermentation broth of high producing strain is processed. In this case, the part of tacrolimus present in the fermentation liquid can be neglected and a simple processing of the mycelium only is acceptable from the viewpoint of yield. The advantage consists in a more simple process and low consumption of solvents as demonstrated by the Example 2. Suitable water miscible solvents for extraction of separated mycelium are preferably acetone, 1-propanol, and/or 2-propanol.

The aqueous extract is further processed without any concentration, what is another advantage of the process. Further processing of the aqueous extract, no matter if the mycelium is separated or not, comprises of the adding a water non miscible solvent to the aqueous extract and mixing the obtained two or three phase system. Tacrolimus and other macrolides are thus extracted into the organic phase, while most ballast components remain in the water phase. Practically any organic water non miscible solvent with the exception of aliphatic hydrocarbons can be used as a suitable water non miscible solvent, but practical reasons (environmental aspects and economical availability) limit the use to some solvents like toluene, xylenes, dichloromethane, dichloroethane, tert-butyl methyl ether, or isobutyl methyl ketone only. Preferred solvent is toluene due to its price, environmental acceptability, a low risk to human health, and other, below discussed aspects. The aim of this operation is not only to purify tacrolimus, but also some concentration of the product, since a very small amount only of toluene can be added to the aqueous extract in order to transfer macrolides into the organic phase quantitatively, as demonstrated in the examples. Another advantage of toluene is simple recovery of the used solvents due to the substantial difference of the boiling points of acetone or 2-propanol, and toluene.

The separated organic extract containing tacrolimus and other macrolides is then concentrated. Another advantage of the use of toluene is evident here. The processes described in the literature require drying of the extracts containing tacrolimus by drying agents. The process according to the invention using toluene as the non water miscible solvent does not require drying. Water present in the organic extract is removed by a simple evaporation as an azeotrope with toluene and dry tacrolimus concentrate is thus obtained.

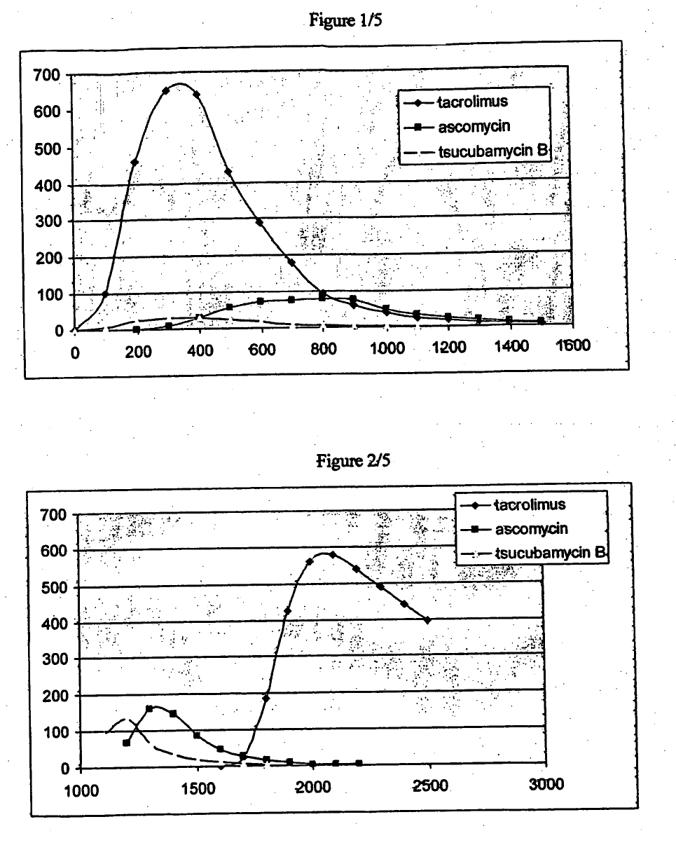

The tacrolimus concentrate obtained according to the invention contains tacrolimus and all other related macrolides present in the fermentation broth, particularly ascomycin and tsucubamycin B. Therefore, further processing must involve separation of tacrolimus from related macrolides. As described above, all the known chromatographic systems utilize a reverse phase chromatography. It was proved by the experimentation that the normal phase chromatography on a silica gel is capable to separate in some extent tacrolimus from ascomycin, but not from tsucubamycin B, as demonstrated on FIG. 1. On the other site, it was found that tacrolimus can be separated from both ascomycin and tsucubamycin B by the normal phase chromatography on the silica gel modified with salts of silver. While ascomycin is more retained that tacrolimus and tsucubamycin B on a silica gel without silver salt, tacrolimus is substantially more retained than both tsucubamycin B and ascomycin on a silver salt modified silica gel, as demonstrated on FIG. 2. The fact that both impurities, ascomycin and tsucubamycin B have shorter retention on the silver salt modified column gives excellent base for the preparative purification of tacrolimus.

The basic principle of the action of silver as the modifier of a silica gel consists in its ability to form complexes with the allyl group of tacrolimus, whereas such group is missing in the structures of other related macrolides. Similarly, some other transition metals, e.g., salts or complexes of platinum group metals, are capable to form η-allyl complexes and act in a similar manner, however, the use of silver as the silica gel modifier is strongly preferred due to its lower price and more simple regeneration. Among suitable silver salts there are binary inorganic salts, e.g., nitrate, fluoride, chlorate, perchlorate, nitrate, or like, and/or organic salts, e.g., acetate, trifluoroacetate, benzoate, cyclohexanebutyrate, acetylacetonate or like, or the salt can be formed by direct bonding to a suitable functional group of a chromatographic sorbent. Since some salts of silver are light sensitive or partly soluble in the mobile phases used for the purification of macrolides, the use of silver nitrate is preferred for its stability.

It was proved by experimentation that suitable solvents for chromatographic separation of tacrolimus from the related macrolide compounds on a silver salt modified silica gel can be different mixtures of commonly used solvents like dichloromethane and its mixture with acetone, isobutyl methyl ketone or tert-butyl methyl ether, or mixtures of toluene with acetone, isobutyl methyl ketone or tert-butyl methyl ether or some esters of aliphatic alcohols with acetic acid e.g., ethyl acetate, propyl acetate, and/or butyl acetate. The preferred solvents are mixtures of toluene with acetone or isobutyl methyl ketone. The separation can be accomplished in the isocratic mode. Then the suitable mobile phase should contain about 15% (v/v) of acetone and about 85% (v/v) of toluene respective about 50% (v/v) of toluene and about 50% (v/v) of isobutyl methyl ketone. Another possibility is to perform the chromatographic purification on a silver salt modified silica gel using gradient mode. Using the above defined preferred solvents means that the chromatography starts, e.g., with pure toluene and the polarity of the mobile phase is stepwise increased by addition of acetone or isobutyl methyl ketone. It is necessary to use the gradient mode when the tacrolimus concentrate is directly loaded on the column. On the other site, the isocratic mode is convenient, when the material loaded on the column was pre-purified so that it does not contain the ballast impurities as described below.

In another embodiment of the invention, the chromatographic purification of the tacrolimus concentrate can be accomplished in two steps, both using normal phase chromatography. In the first step the tacrolimus concentrate is purified on a silica gel, obtaining fraction containing a mixture of macrolides. The sense of this operation is the elimination of ballast impurities other than the macrolides. Then in the second step, the fraction of macrolides from the first chromatography is purified on a silica gel modified with a silver salt. The advantage of such two step purification is the fact that only purified fraction is loaded on the column filled with a silver salt modified silica gel what results in the longer lifetime of the column. Moreover, the second chromatography can be accomplished in isocratic mode, what is very convenient.

Chromatographic separation of tacrolimus from tsucubamycin B and ascomycin on a normal phase using non aqueous solvents as the mobile phase is the basic feature and the main advantage of the process according to the invention. Tacrolimus and other macrolides are relatively unstable. They are prone to isomerisation to, so called, tautomers (tacrolimus tautomer I and tautomer II). This isomerisation is especially rapid in aqueous solutions, used as a mobile phase for reverse phase chromatography. Moreover, the isolation of the product from the eluate obtained from the normal phase chromatography is very simple: the solvent can be evaporated under vacuum, what is not harmful for the product. On the other site, the isolation of the product from the aqueous eluate obtained after reverse phase separation is very difficult and it is usually accompanied by the partial lost of the product.

In another embodiment of the invention, crystalline tacrolimus can be obtained from the chromatographic fractions by crystallization of the residue after evaporation of the mobile phase from a mixture of 2-propanol and water. The crystallization from the mixture of 2-propanol and water can be accomplished by dissolving the residue in 2-propanol and addition of water. It was found out by experimentation that at least one weight part of 2-propanol should be used for dissolving of one weight part of the residue obtained after evaporation of the chromatographic fractions and that the volume ratio of 2-propanol and water should be from about 1:1 to about 1:2. The purification effect of the crystallization can be further improved when some aliphatic hydrocarbon like hexane or heptane is added to the crystallization. The volume of the added aliphatic hydrocarbon is not limited, but it has some impact on the purification effect.

Another suitable solvent for tacrolimus crystallization is diisopropyl ether. The crystallization from this solvent can be accomplished by evaporation of the chromatographic fractions to a dry residue and dissolving the fractions in diisopropyl ether.

### EXAMPLES

The following examples are intended to further illustrate certain preferred embodiment of the invention and are not limiting in nature. Those skilled in the art will recognize, using no more than routine experimentation, numerous equivalents to the specific procedures described herein.

### Example 1

### Isolation of Tacrolimus from Whole Fermentation Broth

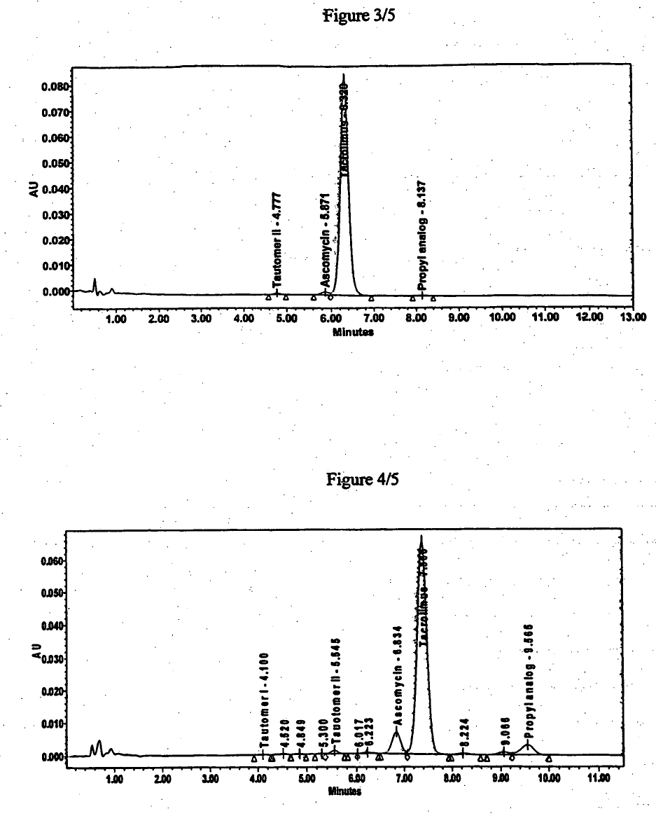

10.0 l of whole fermentation broth obtained by submerged cultivation of Streptomyces sp. producing tacrolimus was diluted with 10.0 l of 2-propanol and the suspension was stirred for 4 hours. Then the solid phase was separated by filtration and the filtrate was extracted two times with 1000 ml of toluene. The pooled toluene extracts were evaporated under reduced pressure to the volume about 25 ml and this concentrate contained 2.12 g of tacrolimus, 0.25 g of ascomycin, and 0.11 g of tsucubamycin B according to the HPLC analysis. The concentrate was loaded on a chromatographic column filled with 200 g of a silica gel (Lichroprep Merck 60,25-40 μm) modified with 20 g of silver nitrate. The column was washed first with toluene (about 400 ml) and then with toluene stepwise polarized with isobutyl methyl ketone, up to 60% (v/v). The fractions containing pure tacrolimus (HPLC monitoring) were pooled and evaporated to dryness and the residue (1.8 g) was crystallized from diisopropyl ether, obtaining 1.1 g of crystalline product, containing according to HPLC analysis 95.8% of tacrolimus, 0.7% of ascomycin, less than 0.1% of tsucubamycin B and about 1% of tacrolimus tautomers—the HPLC record is presented on FIG. 3.

### Example 2

### Isolation of Tacrolimus from Dried Mycelium

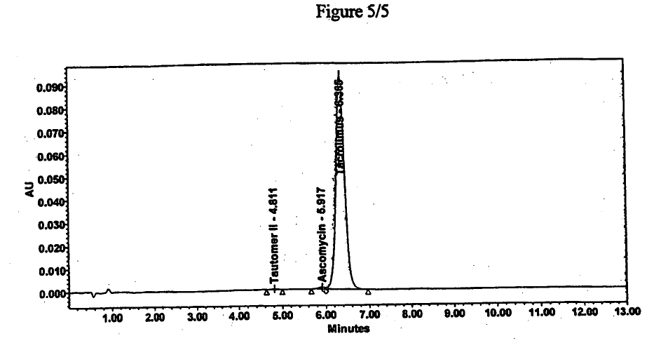

40.0 kg of dry mycelium containing according to HPLC analysis 0.21% of tacrolimus was prepared by processing of 200 l of fermentation broth obtained by submerged cultivation of Streptomyces sp. producing tacrolimus. The mycelium was extracted with 50% (v/v) of acetone, obtaining 40.0 l of the aqueous extract. The aqueous extract was then extracted twice with 4 l of toluene, obtaining 15 l of the organic extract. The organic extract was concentrated to the volume about 1 liter. The concentrate was loaded on a column containing 4.0 kg of a silica gel (Merck 100,63-200 μm). The column was washed first with toluene (about 30 l) and then with toluene stepwise polarized with acetone (up to 20% (v/v) of acetone). The fractions containing tacrolimus (TLC monitoring) were pooled and evaporated to dryness, obtaining residue (residue after first chromatography, 130 g) containing according to HPLC analysis 61.6% of tacrolimus, 7.9% of ascomycin, and 3.5% of tsucubamycin B—the HPLC record of this material is presented on FIG. 4. The residue after first chromatography was further purified by the chromatography on column filled with 1000 g of a silica gel (Lichroprep Merck 60,25-40 μm) modified with 100 g of silver nitrate, using the mixture of toluene and acetone 85:15 (v/v). The chromatographic fractions were monitored by HPLC. Fractions containing less than 0.5% of ascomycin were pooled and concentrated. Fractions containing more than 0.5 and less than 10% ascomycin were recycled. 10 g of the material was purified in one chromatographic run and the chromatography was repeated 17 times (13 times with the concentrate and 4 times with the recycled fractions), using the same column. Finally, 94.9 g of dry residue obtained by concentration of the pooled purified fractions was obtained. The residue was dissolved in 250 ml of 2-propanol and 350 ml of water, 500 ml of n-heptane was added to the solution, and the product was brought to crystallization by cooling and mixing. The crystalline tacrolimus was obtained by the filtration, washing with n-heptane and drying. The product was once more recrystallized from the same solvent mixture obtaining 65.6 g of dry product. According to the HPLC analysis the recrystallized product contained 98.21% of tacrolimus, 0.32% of ascomycin, 0.08% of tsucubamycin B and 0.78% of tacrolimus tautomers—the HPLC record is presented on FIG. 5.

### Example 3

Preparation of the silica gel modified with silver nitrate 10.0 g of crystalline silver nitrate was dissolved in 1000 ml of methanol under heating and 100 g of a silica gel (Lichroprep Merck 60, 25-40 μm) was added to the solution. The suspension was then evaporated to dryness and the residue was dried under vacuum 60 mbar at 70° C.

What we claim is:

1. A process for isolating crystalline tacrolimus from fermentation broth comprising the steps of:
a) preparing of an aqueous extract of tacrolimus
b) extracting of the aqueous extract with a water non miscible solvent at pH from about 5 to about 8 obtaining thus an organic extract and its evaporation obtaining thus tacrolimus concentrate
c) chromatographic purification of the tacrolimus concentrate obtaining thus purified tacrolimus
d) crystallization of the purified tacrolimus from a suitable solvent obtaining crystalline tacrolimus

2. A process of the claim 1 wherein the aqueous extract is prepared by dilution of the fermentation broth with an organic solvent miscible with water.

3. A process of the claim 2 wherein one volume part of the fermentation broth is diluted with at least 0.5 volume part of a water miscible organic solvent.

4. A process of the claim 2 wherein one volume part of the fermentation broth is diluted with maximum two volume parts of a water miscible organic solvent.

5. A process of the claims 1 and 2 wherein the water miscible organic solvent is ethanol, 1-propanol, 2-propanol or acetone, or mixtures thereof.

6. A process of the claims 1 and 2 wherein the aqueous extract is separated from the solid phase by filtration or sedimentation prior to the extraction into the water non-miscible solvent.

7. A process of the claims 1 and 2 wherein the aqueous extract is not separated from the solid phase before the extraction into the water non-miscible solvent.

8. A process of the claim 1 wherein the water non miscible solvent is toluene, xylene, dichloromethane, dichloroethane, tert-butyl methyl ether, methyl isobutyl ketone, or mixtures thereof.

9. A process of the claim 8, wherein the water non-miscible solvent is toluene.

10. A process of the claim 1 wherein the aqueous extract is prepared by separation of the mycelium from the fermentation broth, and its extraction with a mixture of water and an organic solvent miscible with water.

11. A process of the claim 10 wherein the organic solvent miscible with water is ethanol, 1-propanol, 2-propanol, acetone, or mixtures thereof.

12. A process of the claim 10 wherein the mixture of the organic solvent and water used for the extraction contains at least 30% water.

13. A process of the claim 10 wherein the mycelium is extracted with about 50% aqueous solution of acetone.

14. A process of the claim 10 wherein less than 2 liters of aqueous extract are obtained from 1 kilogram of dry mycelium.

15. A process of the claim 1 wherein the chromatographic purification of tacrolimus concentrate is accomplished by normal phase chromatography.

16. A process of the claims 1 and 15 wherein the chromatographic purification is accomplished in two steps using two different chromatographic sorbents.

17. A process of the claims 1 and 15 wherein the chromatographic purification is accomplished in one step.

18. A process of the claims 15 and 16 wherein at least one chromatographic step is accomplished on a silica gel modified with a salt of silver.

19. A process of the claim 18 wherein the salt of silver is silver nitrate.

20. A process of the claim 15 wherein the chromatographic purification of the concentrate is accomplished using organic solvents as the mobile phase.

21. A process of the claim 20 wherein the organic solvent are mixtures of toluene or dichloromethane with isobutyl methyl ketone, acetone or tert-butyl methyl ether or esters of aliphatic alcohols with acetic acid.

22. A process of the claims 20 and 21 wherein the organic solvent is a mixture of toluene and acetone.

23. A process of the claim 1 wherein the purified tacrolimus is crystallized from a mixture of 2-propanol and water.

24. A process of the claim 15 wherein the volume ratio of 2-propanol and water is from 1:1 to 1:2.

25. A process of the claim 23 wherein the crystallization is accomplished by addition of hexane.

26. A process of the claim 25 wherein the quantity of hexane is not limited.

27. A process of the claim 1 wherein the purified tacrolimus is crystallized from diisopropyl ether.
